(19) United States
(12) Patent Application Publication
(10) Pub. No.: US 2017/0233329 A1
(43) Pub. Date: Aug. 17, 2017

(54) PROCESS FOR THE PREPARATION OF AMINO ALCOHOL DERIVATIVES OR SALTS THEREOF

(71) Applicant: GRANULES INDIA LIMITED, HYDERABAD (IN)

(72) Inventors: KRISHNA PRASAD CHIGURUPATI, HYDERABAD (IN); PRASAD RAJU VNKV VETUKURI, HYDERABAD (IN); GOVERDHAN GILLA, HYDERABAD (IN); RAJASEKHAR BHUPATHI, HYDERABAD (IN)

(21) Appl. No.: 15/518,498

(22) PCT Filed: Oct. 26, 2015
(86) PCT No.: PCT/IB2015/058243

§ 371 Date: Apr. 12, 2017

(30) Foreign Application Priority Data
Oct. 27, 2014 (IN) 5320/CHE/2014

(51) Int. Cl. C07C 213/08; C07C 51/42; C07C 51/41; C07D 473/16; C07C 213/10

(52) U.S. Cl. CPC C07C 213/08; C07D 473/16; C07C 213/10; C07C 51/412; C07C 51/42; C07C 2601/10

(57) ABSTRACT

The present invention relates to a process for the preparation of amino alcohol derivatives or salts thereof. In particular the present invention relates to process for the preparation of amino alcohol derivatives or salts thereof which may be used as intermediates in the preparation of HIV reverse transcriptase inhibitors, more preferably Carbovir and Abacavir.

The present invention more specifically relates to a process for the preparation of (1S,4R)-4-amino-2-cyclopentene-1-methanol of Formula IIIa.

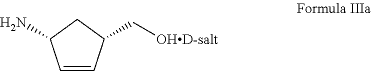

The present invention also specifically relates to process for the preparation of Abacavir sulfate of Formula II using compound of Formula IIIa prepared according to the process of the present invention.

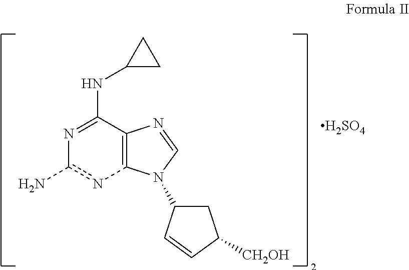

### FIELD OF THE INVENTION

The present invention relates to a process for the preparation of amino alcohol derivatives or salts thereof. In particular the present invention relates to process for the preparation of amino alcohol derivatives or salts thereof which may be used as intermediates in the preparation of HIV reverse transcriptase inhibitors, more preferably Carbovir and Abacavir.

The present invention more specifically relates to a process for the preparation of (1S,4R)-4-amino-2-cyclopentene-1-methanol of Formula IIIa.

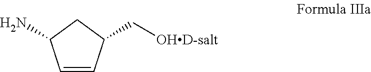

The present invention also specifically relates to process for the preparation of Abacavir sulfate of Formula II using compound of Formula IIIa prepared according to the process of the present invention.

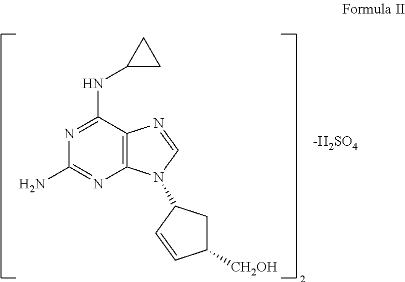

### BACKGROUND OF THE INVENTION

Acquired immune deficiency syndrome (AIDS) has rapidly become one of the major causes of death in the world. It is estimated that over 40 million people are infected with the human immunodeficiency virus (HIV), which is the causative agent of AIDS. In 1985, 3′-azido-3′-deoxythymidine (AZT) was approved as the first synthetic nucleoside to inhibit the replication of HIV. Since then, a number of other synthetic nucleoside analogs have been proven to be effective against HIV. After cellular phosphorylation to the triphosphate form by cellular kinases, the nucleotides are incorporated into a growing strand of viral DNA and cause chain termination due to the absence of the 3′-hydroxyl group.

Carbocyclic nucleosides are structural analogs to nucleosides in which the furanose oxygen is replaced by a methylene group. Similar to native nucleosides, carbocyclic nucleosides can behave as inhibitors of the enzymes. However, because carbocyclic nucleosides lack the labile glycosidic linkage between heterocycle and sugar of native nucleosides, they are not susceptible to hydrolysis by phosphorylases or phosphotransferases.

Carbocyclic nucleosides have been the subject of extensive investigation because of the variety of biological properties displayed by these compounds. Of particular interest is the potential of carbocyclic nucleosides for use in antiviral, antitumor and anticancer chemotherapeutic applications. Perhaps the best known examples of such carbocyclic nucleosides are Abacavir and Carbovir, both of which show great promise as anti-HIV agents.

Carbovir has been reported as the first carbocylic nucleoside analogue, with potent anti-HIV activity in vitro; its discovery provided a base for the synthesis of other carbocyclic analogues. The first synthesis of Carbovir has been accomplished in 1990 by Vince et al., as a racemic mixture of two enantiomers. Afterwards, a chemoenzymatic synthesis of both enantiomers of Carbovir, has been reported. The natural (−)-enantiomer of Carbovir is primarily responsible for the antiviral activity.

Chemically Carbovir is represented as 2-Amino-9-[(1R,4S)-4-(hydroxymethyl)-2-cyclopenten-1-yl]-1,9-dihydro-6H-purin-6-one and structurally as shown below:

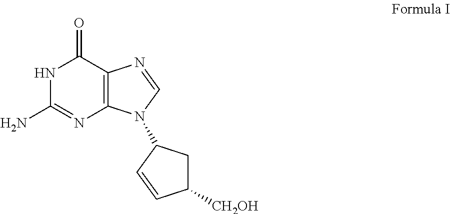

Abacavir sulphate, having the brand name ZIAGEN®, is a synthetic carbocyclic nucleoside analogue with inhibitory activity against HIV-1. The chemical name of Abacavir sulfate is (1S,cis)-4-[2-amino-6-(cyclopropylamino)-9H-purin-9-yl]-2-cyclopentene-1-methanol sulfate (salt) (2:1). Abacavir sulfate is the enantiomer with 1S, 4R absolute configuration on the cyclopentene ring. It has a molecular formula of (C₁₄H₁₈N₆O)₂.H₂SO₄ and a molecular weight of 670.76 daltons. It has the following structural formula:

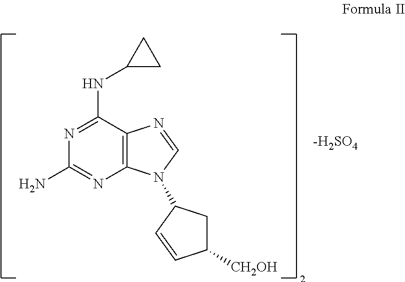

U.S. Pat. No. 5,034,394 discloses a process for the preparation of (1S,4R)-4-amino-2-cyclopentene-1-methanol dibenzoyl-D-tartrate which comprises reaction of racemic 2-azabicyclo[2.2.1]hept-5-en-3-one with hydrogenchloride in methanol to give (±)cis-methyl-4-amino-2-cyclopentene-1-carboxylate hydrochloride which on reduction with DIBAL-H in hexane gave racemic 4-amino-2-cyclopentene-1-methanol which is further subjected to resolution with dibenzoyl-D-tartaric acid to give (1S,4R)-4-amino-2-cyclopentene-1-methanol dibenzoyl-D-tartrate. The process is shown in the scheme given below:

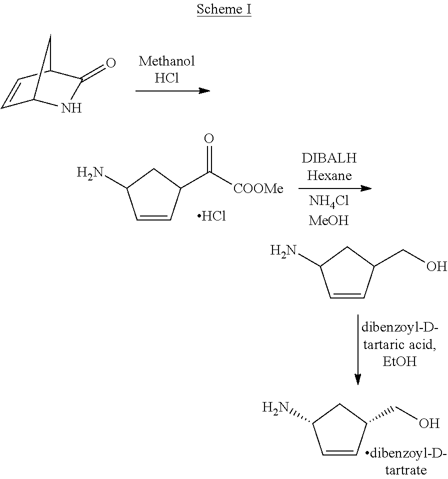 [SMILES: O=C1NC2C=CC1C2 ; COC(=O)C(=O)C1C=CC(N)C1 ; Cl ; NC1C=CC(CO)C1 ; N[C@H]1C=C[C@@H](CO)C1]

U.S. Pat. No. 6,448,402 discloses a process for the preparation of compound of (1S,4R)-4-amino-2-cyclopentene-1-methanol D-hydrogen tartrate which comprises resolution of racemic 1-amino-4-(hydroxymethyl)-2-cyclopentene with D(−)-tartaric acid in methanol at reflux temperature followed by cooling to 20° C. for about 2 hours gave crystals of compound of (1S,4R)-4-amino-2-cyclopentene-1-methanol D-hydrogen tartrate, Racemic 1-amino 1 (hydroxymethyl)-2-cyclopentene is prepared in turn by reduction of 2-azabicyclo[2.2.1]hept-5-en-3-one using lithium borohydride. The process is shown in the scheme given below:

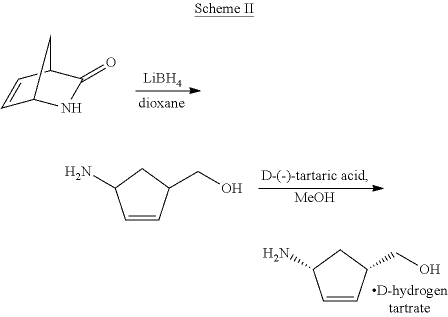

U.S. Pat. No. 6,495,711 discloses a process for the preparation of compound of (1S,4R)-4-amino-2-cyclopentene-1-methanol L-hydrogen tartrate which comprises resolution of racemic reacting racemic 2-azabicyclo[2.2.1]hept-5-en-3-one with methanol in the presence of HCl gas followed by resolution of the obtained compound with L(+)-tartaric acid in water, addition of triethylamine and then workup resulted in crystals of (1S,4R)-methyl-4-amino-2-cyclopentene carboxylate L-hydrogen tartrate. The process is shown in the scheme given below:

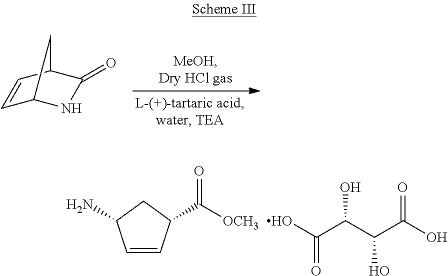

Nucleosides, Nucleotides & Nucleic acid, 19(1&2), 297-327, 2000 discloses a process for the preparation of (1S,4R)-4-amino-2-cyclopentene-1-methanoldibenzoyl-D-tartrate which comprises reduction of tosylate salt of 4-amino-2-cyclopentene-1-carboxylate using lithium aluminium hydride in THF to give racemic 4-amino-2-cyclopentene-1-methanol followed by resolution of the same with dibenzoyl-D-tartaric acid in ethanol and acetonitrile to give (1S,4R)-4-amino-2-cyclopentene-1-methanol dibenzoyl-D-tartrate. The process is shown in the scheme given below:

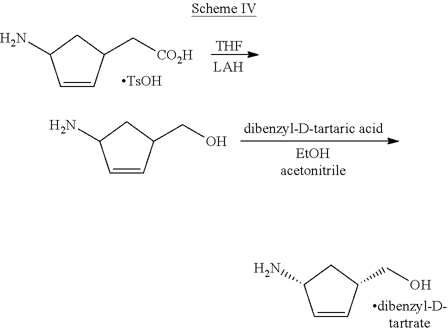

The methods for preparing compound of (1S,4R)-4-amino-2-cyclopentene-1-methanol or its salts shown in the above prior art are disadvantageous in that they employ expensive, toxic reagents and require extreme reaction conditions, multiple steps thereby rendering them unsuitable for bulk production.

Accordingly, there has been a need to develop a simple and industrially viable process for preparing (1S,4R)-4-amino-2-cyclopentene-1-methanol or its salt of compound of Formula III by using intermediate of Formula IV.

### OBJECTIVES OF THE INVENTION

The main objective of the present invention is to provide a process which is safe on industrial scale for the preparation of (1S,4R)-4-amino-2-cyclopentene-1-methanol salt of Formula IIIa.

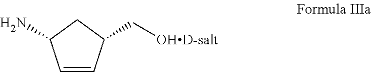

Another objective of the present invention is to provide a commercially viable process for the preparation of compound of Formula IIIa through intermediate compound of Formula IVa.

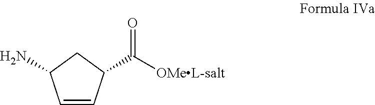

Yet another objective of the present invention is to provide industrially safe and commercially viable process for the preparation of compound of (1S,4R)-4-amino-2-cyclopentene-1-methanol D-hydrogen tartrate of Formula IIIb.

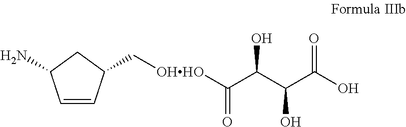

Yet another objective of the present invention of is to provide a process for the preparation of Carbovir and Abacavir using (1S,4R)-4-amino-2-cyclopentene-1-methanol salt prepared according to the process of the present invention.

Yet another objective of the present invention provides an improved process for the preparation of (1S,4R)-4-amino-2-cyclopentene-1-methanol of Formula III.

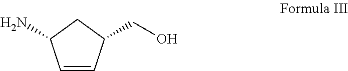

Yet another objective of the present invention provides an improved process for the preparation of (1S,4R)-methyl-4-amino-2-cyclopentene carboxylate L-hydrogen tartrate of Formula IVb.

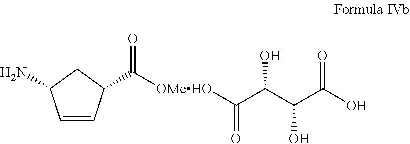

Yet another objective of the present invention provides an improved process for the preparation of (1S,4R)-methyl-4-amino-2-cyclopentene carboxylate of Formula IV.

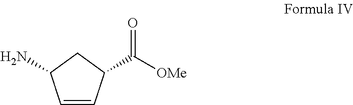

### SUMMARY OF THE INVENTION

Accordingly, the present invention provides an improved process for the preparation of (1S,4R)-4-amino-2-cyclopentene-1-methanol salt of Formula IIIa

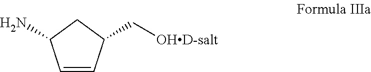

which comprises the steps of
i) hydrolysis of compound of formula IVa

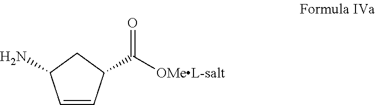

in the presence of a base.
ii) reduction of the compound obtained in step i) using a suitable reducing agent in a solvent,
iii) resolution of compound obtained in step ii) using a chiral acid with D-configuration in a solvent and
iv) isolating compound of Formula IIIa.

Another embodiment of the present invention provides an improved process for the preparation of (1S,4R)-4-amino-2-cyclopentene-1-methanol salt of Formula IIIa

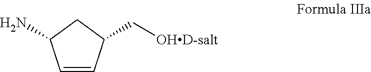

which comprises the steps of

i) converting of compound of formula IVa

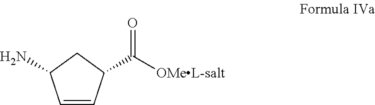

to compound of Formula IIIc

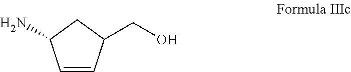

using a suitable reducing agent in the presence of a base and in a solvent,
iii) resolution of compound obtained in step i) using a chiral acid with D-configuration in a solvent and
iv) isolating compound of Formula IIIa.

In another embodiment, the present invention provides an improved process for the preparation of (1S,4R)-4-amino-2-cyclopentene-1-methanol of Formula III

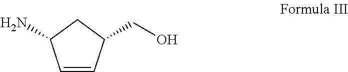

which comprises the steps of reduction of compound of Formula IV or its salts

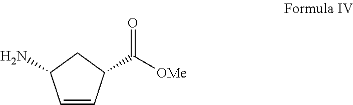

using a suitable reducing agent in the presence of a base and in a solvent to compound of Formula III or its salts.

In one embodiment, the present invention provides an improved process for the preparation of (1S,4R)-4-amino-2-cyclopentene-1-methanol of Formula III,

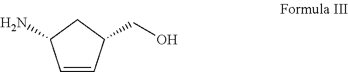

which comprises the steps of
i) hydrolysis of compound of formula IVa

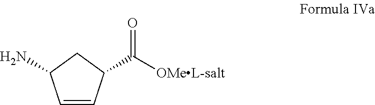

in the presence of a base,
ii) reduction of the compound obtained in step i) using a suitable reducing agent in a solvent,
iii) resolution of compound obtained in step ii) using a chiral acid with D-configuration in a solvent and
iv) converting the compound obtained in step iii) to compound of Formula III.

In a preferred embodiment, the present invention provides an improved process for the preparation of (1S,4R)-4-amino-2-cyclopentene-1-methanol of Formula III

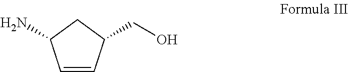

which comprises the steps of
i) hydrolysis of compound of formula IVb

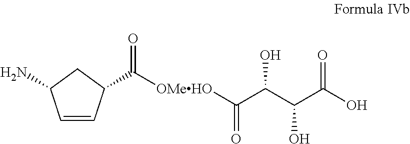

in the presence of sodium hydroxide.
ii) reduction of the compound obtained in step i) using sodium borohydride in 2-butanol,
iii) resolution of compound obtained in step ii) using D-tartaric acid in methanol and
iv) converting the compound obtained in step iii) to compound of Formula III.

In another preferred embodiment, the present invention provides an improved process for the preparation of (1S,4R)-4-amino-2-cyclopentene-1-methanol D-hydrogen tartrate of Formula IIIb which comprises the steps of

i) hydrolysis of compound of formula IVb

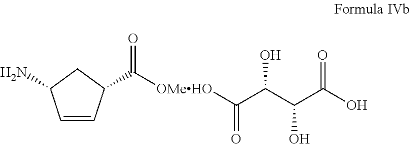

in the presence of a base,
ii) reduction of the compound obtained in step i) using a suitable reducing agent in a solvent,
iii) resolution of compound obtained in step ii) using a chiral acid with D-configuration in a solvent and
iv) isolating compound of Formula IIIb.

In a most preferred embodiment, the present invention provides an improved process for the preparation of (1S,4R)-4-amino-2-cyclopentene-1-methanol D-hydrogen tartrate of Formula IIIb which comprises the steps of

i) hydrolysis of compound of formula IVb

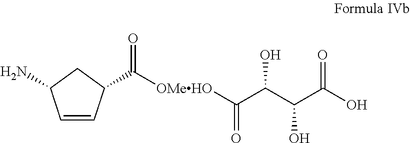

in the presence of sodium hydroxide,
ii) reduction of the compound obtained in step i) using sodium borohydride in 2-butanol,
iii) resolution of compound obtained in step ii) using D-tartaric acid in methanol and
iv) isolating compound of Formula IIIb.

In another most preferred embodiment, the present invention provides an improved process for the preparation of (1S,4R)-4-amino-2-cyclopentene-1-methanol D-hydrogen tartrate of Formula IIIb which comprises the steps of

i) reaction of racemic 2-azabicyclo[2.2.1]hept-5-en-3-one

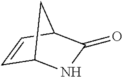

with alcohol in the presence of an acid followed by resolution using a chiral acid with L-configuration to give (1S,4R)-methyl-4-amino-2-cyclopentene carboxylate of Formula IVa,

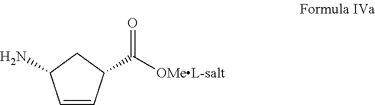

ii) hydrolysis of compound of formula IVa in the presence of a base,
iii) reduction of the compound obtained in step ii) using a suitable reducing agent in a solvent,
iv) resolution of compound obtained in step iii) using a chiral acid with D-configuration in a solvent and
v) isolating compound of Formula IIIb.

In yet another most preferred embodiment, the present invention provides an improved process for the preparation of (1S,4R)-4-amino-2-cyclopentene-1-methanol D-hydrogen tartrate of Formula IIIb which comprises the steps of:

i) reaction of racemic 2-azabicyclo[2.2.1]hept-5-en-3-one

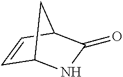

with methanol in the presence of dry HCl gas followed by resolution with L-(+)-tartaric acid in water in the presence of triethylamine give (1S,4R)-methyl-4-amino-2-cyclopentene carboxylate L-hydrogen tartrate of Formula IVb,

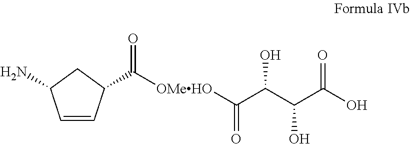

ii) hydrolysis of compound of formula IVb in the presence of sodium hydroxide,
iii) reduction of the compound obtained in step ii) using sodium borohydride in 2-butanol,
iv) resolution of compound obtained in step iii) using D-tartaric acid in methanol and
v) isolating compound of Formula IIIb.

The (1S,4R)-4-amino-2-cyclopentene-1-methanol of Formula III prepared according to the process of the invention is converted to Carbovir(I) or Abacavir (II) which comprises the steps of:

i) reaction of compound of Formula V

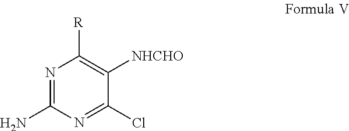

wherein R is halogen selected from chloro, fluoro, bromo, iodo; with compound of Formula IIIa

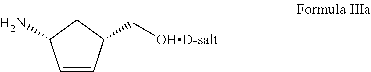

in a solvent in the presence of a base to give compound of Formula VI,

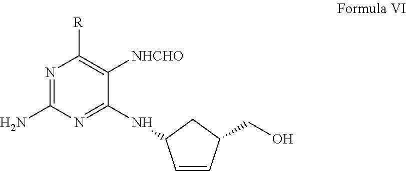

wherein R is halogen selected from chloro, fluoro, bromo, iodo;
ii) cyclization of compound of Formula VI in the presence of triethyl orthoformate to give compound of Formula VII,

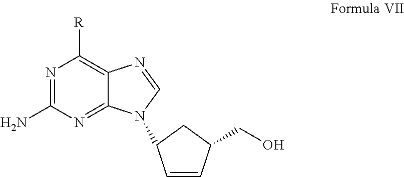

wherein R is halogen selected from chloro, fluoro, bromo, iodo,
iii) conversion of compound of Formula VII to compound of Formula I or II where in R is OH and cyclopropylamine respectively.

In another preferred embodiment, the present invention provides an improved process for the preparation of (1S,4R)-4-amino-2-cyclopentene-1-methanol of Formula III

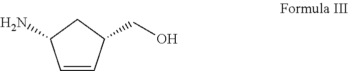

which comprises the steps of reducing the compound of Formula IV or its salts

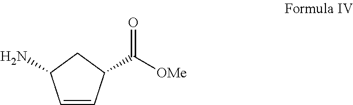

using sodium borohydride to compound of Formula III or its salts.

### DETAILED DESCRIPTION OF THE INVENTION

Suitable base used in the hydrolysis is selected from alkali metal hydroxides such as sodium hydroxide, lithium hydroxide or potassium hydroxide, or alkali metal carbonates such as caesium carbonate, sodium carbonate or potassium carbonate, or alkoxides such as sodium methoxide, potassium methoxide, sodium ethoxide, potassium ethoxide, the base optionally being present in aqueous solution.

Suitable reducing agents are selected from metal hydrides such as boron reagents like sodium borohydride, lithium borohydride, zinc borohydride, sodium cyanoborohydride, sodium triacetoxyborohydride, lithium cyanoborohydride and the like or aluminum reagents like diisobutylaluminum hydride, aluminum hydride, lithium aluminum hydride and the like.

Suitable solvents used in reduction are selected from solvents that are inert to the reaction. Examples of such solvents include alcohols such as methanol, ethanol, propanol, butanol and the like or aromatic hydrocarbons such as benzene, toluene, xylene, chlorobenzene and the like or aliphatic hydrocarbons such as heptane, hexane and the like or halogenated hydrocarbons such as chloroform, dichloromethane and the like or ethers such as diethyl ether, tetrahydrofuran, dioxane and the like and or mixtures thereof.

Chiral acid used for resolution are selected from acids having D-configuration such as malic acid, mandelic acid, 2-chloromandelic, 3-chloromandelic, 4-chloromandelic acid, tartaric acid, diacetyl tartaric acid, di-p-anisolyl tartaric acid, dibenzoyl tartaric acid, ditoluoyl tartaric acid, camphorsulfonic acid.

Suitable solvents used in resolution are selected from alcohols such as methanol, ethanol, isopropanol, butanol, 1,2-dimethoxy ethanol, 2-methoxy ethanol, 2-ethoxy ethanol and ethylene glycol; ethers such as diethyl ether, 1,4-dioxane, dimethoxy ethane, DIPE, MTBE, THF, 2-methyl tetrahydrofuran and aprotic polar solvents such as DMF, DMSO, DMA and or mixtures thereof.

The (1S,4R)-4-amino-2-cyclopentene-1-methanol of Formula III prepared according to the process of the invention is converted to Carbovir(I) or Abacavir (II) which comprises the steps of:

i) reaction of compound of Formula V

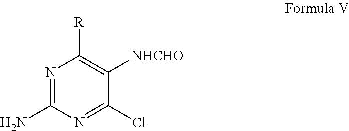

wherein R is halogen selected from chloro, fluoro, bromo, iodo; with compound of Formula IIIa

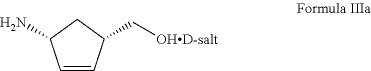

in a solvent in the presence of a base to give compound of Formula VI,

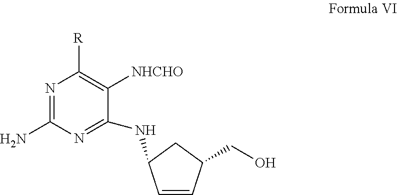

wherein R is halogen selected from chloro, fluoro, bromo, iodo;
ii) cyclization of compound of Formula VI in the presence of triethyl orthoformate to give compound of Formula VII,

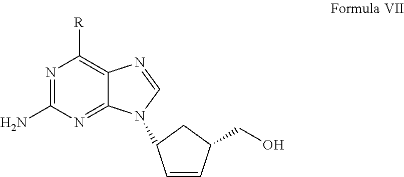

wherein R is halogen selected from chloro, fluoro, bromo, iodo;
iii) conversion of compound of Formula VII to compound of Formula I or II where in R is OH and cyclopropylamine respectively.

Suitable base used in step i) is selected from organic bases such as triethylamine, tributylamine, N-methylmorpholine, N,N-diisopropylethylamine, N-methylpyrrolidine, pyridine, 4-(N,N-dimethylamino) pyridine, morpholine, imidazole, 2-methylimidazole, 4-methylimidazole, or the like; inorganic bases such as alkali metal hydrides like lithium hydride, sodium hydride, potassium hydride, or the like; sodamide; n-butyl lithium; lithium diisopropylamide; alkali metal hydroxides, such as lithium hydroxide, sodium hydroxide, potassium hydroxide, or cesium hydroxide; alkaline metal hydroxides, such as aluminum hydroxide, magnesium hydroxide, calcium hydroxide, or the like; alkali metal carbonates, such as sodium carbonate, potassium carbonate, lithium carbonate, cesium carbonate, or the like; alkaline earth metal carbonates such as magnesium carbonate, calcium carbonate, or the like; alkali metal bicarbonates, such as lithium bicarbonate, sodium bicarbonate, potassium bicarbonate, or the like.

Suitable solvents used in step i) is selected from water; alcohols, such as for example, methanol, ethanol, propanol, butanol, pentanol, isopropyl alcohol, 2-butanol, ethylene glycol, glycerol, or the like; esters, such as for example, ethyl formate, methyl acetate, ethyl acetate, propyl acetate, butyl acetate, methyl propanoate, ethyl propanoate, methyl butanoate, ethyl butanoate, or the like; aromatic hydrocarbons, such as for example, toluene, xylene, chlorobenzene, tetralin, or the like; nitriles, such as for example, acetonitrile, propionitrile, or the like; or any mixtures thereof

The following examples describes the nature of the invention and are given only for the purpose of illustrating the present invention in more detail and are not limitative and relate to solutions which have been particularly effective on a bench scale.

### EXAMPLES

### Example 1: Preparation of (1S,4R)-methyl 4-aminocyclopent-2-enecarboxylate-L-hydrogen tartrate

To a solution of methanolic hydrochloride was added vince lactum (0.9163 mol) in methanol at room temperature and stirred for 30 min. Distilled out methanol completely. Allowed to cool to room temperature, then water and L-(+)-tartaric acid (0.5496 mol) was added. Adjusted the pH to 4.4-4.5 with triethylamine and maintained for about 5-6 hrs at RT. Filtered and washed with methanol to obtain pure compound.

### Example 2: Preparation of (1S,4R)-methyl 4-aminocyclopent-2-enecarboxylate-L-hydrogen tartrate

To methanol (1V) was added vince lactum (0.916 mol) and cooled to 10-15° C. Thionyl chloride was added dropwise up to 30° C. Maintained for 10-15 mm. Distilled out methanol completely. Allowed the mass to room temperature, then added methanol (1.5V), water and L-(+)-tartaric acid (0.549 mol). pH was adjusted to 4.0-4.5 with triethylamine. Maintained for 30 min at room temperature and 30 min at 65° C. Cooled to room temperature and maintained the same temperature for 1 hr. Filtered the solid & washed with methanol. Purified in methanol to obtain pure compound. (Yield: 78%, HPLC purity: 99.69%).

### Example 3: Preparation of ((1S,4R)-4-aminocyclopent-2-enyl)methanol-D-hydrogen tartrate

To a solution of aq.sodium hydroxide was added sodium borohydride (0.9231 mol) and cooled to −5 to 0° C. (1S,4R)-methyl 4-aminocyclopent-2-enecarboxylate-L-hydrogen tartrate (0.39/18) was added and maintained the reaction mass at the same temperature for about 8 hrs. Adjusted the pH to 12-13 with aq.sodium hydroxide solution. Extracted the reaction mass with n-butanol. Butanol was completely distilled out below 60° C. then added methanol and D-(−)-tartaric acid. Filtered the obtained reaction mass, charged MLs in to RBF, heated to 60-65° C. and maintained for 30 min. Then cooled to 15-20° C. and maintained for 6 hours at 15-20° C. Filtered the mass and washed the cake with methanol. The compound was confirmed by ¹H-NMR, Mass and SOR, [α]²⁵_{D} (0.5 in methanol)=−28.3°.

### Example 4: Preparation of ((1S,4R)-4-aminocyclopent-2-enyl)methanol-D-hydrogen tartrate

To a solution of Aq. Sodium hydroxide was added sodium borohydride (0.8000 mol) and cooled to −5 to 0° C. (1S,4R)-methyl 4-aminocyclopent-2-enecarboxylate-L-hydrogen tartrate (0.343 mol) was added at −5 to 0° C. and maintained for 4 hrs at −5 to 0° C. Adjusted the pH to 12-13 with aq.sodium hydroxide solution. Extracted the reaction mass with n-butanol at room temperature. Butanol was completely distilled out below 50° C. under vacuum then added methanol and D-(−)-tartaric acid. Heated the mass to 65° C., maintained for 30 min and concentrated the mass to half volume. Allowed the mass to room temperature, and maintained for 3 hrs. Then filtered the mass and washed the cake with methanol. (Yield: 88%, HPLC purity: 99.91%).

### Example 5: Preparation of ((1S,4R)-4-aminocyclopent-2-enyl)methanol hydrochloride

To a solution of Aq. Sodium hydroxide was added sodium borohydride (1.008 mol) and cooled to −5 to 0° C. (1S,4R)-methyl 4-aminocyclopent-2-enecarboxylate-L-hydrogen tartrate (0.429 mol) was added at −5 to 0° C. and maintained for 4 hrs at −5 to 0° C. Adjusted the pH to 12-13 with aq.sodium hydroxide solution. Extracted the reaction mass with n-butanol at room temperature. Butanol was completely distilled out below 50° C. under vacuum. Isopropyl alcohol was added and cooled to 0-5° C. then added IPA.HCl at the same temperature. Heated the mass to 80-85° C. and maintained for 30 min. Allowed the mass to room temperature, and further cooled to 0-5° C. maintained for 2 hrs. Then filtered the mass and washed the cake with isopropyl alcohol. (Yield: 82%, HPLC purity: 99.59%).

### Example 6: Preparation of N-[2-Amino-4-chloro-6-[[(1R,4S)-4-(hydroxymethyl)-2-cyclopenten-1-yl]amino]-5-pyrimidinyl]formamide

To isopropyl alcohol was added ((1S,4R)-4-aminocyclopent-2-enyl)methanol-D-hydrogen tartrate (0.5360 mol) and sodium carbonate (1.2 mol), stirred for 30 min at room temperature. Then added N-(2-amino-4,6-dichloro-5-pyrimidinyl)formamide (0.4830 mol) and refluxed for 24 hrs. Cool the reaction mass to 50-55° C. Filtered the mass, concentrated the mass and purified in isopropyl alcohol.

### Example 7: Preparation of N-[2-Amino-4-chloro-6-[[(1R,4S)-4-(hydroxymethyl)-2-cyclopenten-1-yl]amino]-5-pyrimidinyl]formamide

To isopropyl alcohol (10V) was added ((1S,4R)-4-aminocyclopent-2-enyl)methanol hydrochloride (0.057 mol) and sodium bicarbonate (0.094 mol), stirred for 60 min at room temperature. Then added N-(2-amino-4,6-dichloro-5-pyrimidinyl)formamide (0.048 mol) and refluxed for 24-26 hrs. Cool the reaction mass to 70-75° C. Filtered the mass, wash the cake with isopropyl alcohol (1V) and concentrated the mass under vacuum below 55° C. up to 3.0-3.5 V. Cooled the mass to room temperature and further cooled to 0-5° C. and stirred for 1 hr. Filtered the solid & washed with isopropyl alcohol. (Yield: 51.4%, HPLC purity: 98.66%).

### Example 8: Preparation of N-[2-Amino-4-chloro-6-[[(1R,4S)-4-(hydroxymethyl)-2-cyclopenten-1-yl]amino]-5-pyrimidinyl]formamide

To isopropyl alcohol (4V) was added ((1S,4R)-4-aminocyclopent-2-enyl)methanol hydrochloride (0.0289 mol) and triethyl amine (6V), stirred for 60 min at room temperature. Then added N-(2-amino-4,6-dichloro-5-pyrimidinyl) formamide (0.024 mol) and refluxed for 3-4 hrs. Cool the reaction mass to 70-75° C. Filtered the mass, wash the cake with isopropyl alcohol (1V) & concentrated the mass under vacuum below 55° C. up to 3.0-3.5 V. Cooled the mass to room temperature and further cooled to 0-5° C. and stirred for 1 hr. Filtered the solid and washed with isopropyl alcohol. ((Yield: 44.1%, HPLC purity: 98.69%).

### Example 9: Preparation of N-[2-Amino-4-chloro-6-[[(1R,4S)-4-(hydroxymethyl)-2-cyclopenten-1-yl]amino]-5-pyrimidinyl]formamide

To isopropyl alcohol (15V) was added ((1S,4R)-4-aminocyclopent-2-enyl)methanol-D-hydrogen tartrate (0.5323 mol) and sodium carbonate (1.8867 mol), stirred for 60 min at room temperature. Then added N-(2-amino-4,6-dichloro-5-pyrimidinyl) formamide (0.4830 mol) and refluxed for 10-11 hrs. Cool the reaction mass to 70-75° C. Filtered the mass, wash the cake with isopropyl alcohol (1V) & concentrated the mass under vacuum below 55° C. Charged isopropyl alcohol (2.5-3.0 V) at 40° C. Maintained the mass for 1 hr at room temperature and 1 hr at 0-5° C. Filtered the solid & washed with isopropyl alcohol. ((Yield: 88.2%, HPLC purity: 98.93%).

### Example 10: Preparation of N-[2-Amino-1-chloro-6-[[(1R,1S)-1-(hydoxymethyl-2-cyclopenten-1-yl]amino]-5-pyrimidinyl]formamide

To isopropyl alcohol (5V) and D.M. water (1.5V) was added ((1S,4R)-4-aminocyclopent-2-enyl)methanol-D-hydrogen tartrate (0.5071 mol) and sodium carbonate (1.0144 mol) lot wise in 20-30 min, stirred for 10 min at room temperature. Then added N-(2-amino-4,6-dichloro-5-pyrimidinyl) formamide (0.4830 mol) and refluxed for 4-5 hrs. Cool the reaction mass to 70-75° C. Filtered the mass, wash the cake with isopropyl alcohol (4V) & concentrated the mass under vacuum below 70° C. Charged D.M. water (5V) at 50° C. Maintained the mass for 2-3 hrs at room temperature. Filtered the solid and washed with water and isopropyl alcohol. (Yield: 80%, HPLC purity: 99.14%).

### Example 11: Preparation of ((1S,4R)-4-(2-amino-6-chloro-9H-purin-9-yl)cyclopent-2-enyl)methanol Hydrochloride

N-[2-amino-4-chloro-6-[[(1R,4S)-4-(hydroxymethyl)-2-cyclopenten-1-yl]amino]-5-pyrimidinyl]formamide (0.3527 mol) was added to trietyl orthoformate (4.2 mol) and hydrochloride acid at 10-15° C. Maintained for 24 hrs at room temperature. Filtered the mass and purified in methanol.

### Example 12: Preparation of ((1S,4R)-4-(2-amino-6-chloro-9H-purin-9-yl)cyclopent-2-enyl)methanol Hydrochloride

N-[2-Amino-4-chloro-6-[[(1R,4S)-4-(hydroxymethyl)-2-cyclopenten-1-yl]amino]-5-pyrimidinyl]formamide (0.1762 mol) was added to trietylorthoformate (7V). Cooled the mass to 0-5° C., hydrochloride acid (0.7V) was added at 0-10° C. After addition maintained the mass for 60 min at 0-5° C. Allowed the mass to room temperature and maintained for 16-18 hrs at. Filtered the mass and purified in methanol. (Yield: 76%, HPLC purity: 98.69%).

### Example 13: Preparation of {(1S,4R)-4-[2-amino-6-(cyclopropylamino)-9H-purin-9-yl]cyclopent-2-en-1-yl}methanol

To isopropyl alcohol was added ((1S,4R)-4-(2-amino-6-chloro-9H-purin-9-yl)cyclopent-2-enyl)methanol Hydrochloride (0.3309 mol), triethyl amine (0.9921 mol) and cyclopropylamine (0.6617 mol) at room temperature. Heated the mass to reflux and maintained for 12 hrs at reflux. Distilled off isopropyl alcohol completely then water and ethyl acetate was added. The reaction mass pH was adjusted to basic with aq.sodium hydroxide solution. Separated the organic layer and aq.layer was extracted twice with ethyl acetate. Ethyl acetate was concentrated and the crude product was isolated in acetone to obtain pure compound. The compound was confirmed by ¹H-NMR, Mass and IR. (Yield: 86%, HPLC >99.2%).

### Example 14: Preparation of {(1S,4R)-4-[2-amino-6-(cyclopropylamino)-9H-purin-9-yl]cyclopent-2-en-1-yl}methanol

To isopropyl alcohol was added ((1S,4R)-4-(2-amino-6-chloro-9H-purin-9-yl)cyclopent-2-enyl)methanol Hydrochloride (0.3309 mol), triethyl amine (0.9921 mol) and cyclopropyl amine (0.6617 mol) at room temperature. Heated the mass to reflux and maintained for 12 hrs at reflux. Distilled off isopropyl alcohol completely then water and toluene were added. Filtered the mass and purified in acetone to obtain pure compound.

### Example 15: Preparation of {(1S,4R)-4-[2-amino-6-(cyclopropylamino)-9H-purin-9-yl]cyclopent-2-en-1-yl}methanol

To isopropyl alcohol was added ((1S,4R)-4-(2-amino-6-chloro-9H-purin-9-yl)cyclopent-2-enyl)methanol Hydrochloride (0.3309 mol), triethyl amine (0.9921 mol) and cyclopropyl amine (0.6617 mol) at room temperature. Heated the mass to reflux and maintained for 12 hrs at reflux. Distilled off isopropyl alcohol completely then water and cyclohexane was added. Filtered the mass and purified in acetone to obtain pure compound.

### Example 16: Preparation of {(1S,4R)-4-[2-amino-6-(cyclopropylamino)-9H-purin-9-yl]cyclopent-2-en-1-yl}methanol

To isopropyl alcohol, triethyl amine (0.252 mol) was added ((1S,4R)-4-(2-amino-6-chloro-9H-purin-9-yl)cyclopent-2-enyl)methanol Hydrochloride (0.082 mol). Raised the mass temperature to 40-45° C. and cyclopropyl amine (0.165 mol) was added at this temp. Heated the mass to reflux and maintained for 8-9 hrs at reflux. Isopropyl alcohol was completely distilled and water & ethyl acetate was added. The reaction mass pH was adjusted to to 9.5-10.0 with ammonium hydroxide solution. Separated the organic layer and aq. layer was extracted twice with ethyl acetate. Ethyl acetate was concentrated and the crude product was isolated in acetone to obtain pure compound. (Yield: 82%, HPLC purity: 99.28%).

### Example 17: Preparation of {(1S,4R)-4-[2-amino-6-(cyclopropylamino)-9H-purin-9-yl]cyclopent-2-en-1-yl}methanol

To water (3V), triethyl amine (0.3458 mol) was added al S,4R)-4-(2-amino-6-chloro-9H-purin-9-yl)cyclopent-2-enyl)methanol Hydrochloride (0.1158 mol). Stirred for 5-10 min and added cyclopropyl amine (0.1754 mol) at room temperature. Heated the mass to 70-75° C. and maintained for 2-3 hrs. Cooled to room temp. The reaction mass pH was adjusted to 9.5-10.0 with ammonium hydroxide solution. The mass was extracted in to MDC and methanol mixture and was given charcoal treatment for two times. The solvent was concentrated below 55° C. and the crude product was isolated in acetone to obtain pure compound. (Yield: 90%, HPLC purity: 99.57%).

### Example 18: Preparation of {(1S,4R)-4-[2-amino-6-(cyclopropylamino)-9H-purin-9-yl]cyclopent-2-en-1-yl}methanol sulphate

To a solution of isopropyl alcohol and water was added {(1S,4R)-4-[2-amino-6-(cyclopropylamino)-9H-purin-9-yl]cyclopent-2-en-1-yl}methanol (0.3492 mol). Heated to 50-55° C. Activated carbon was added and filtered through hyflo bed. To the filter MLs conc. Sulphuric acid (0.2 mol) was added drop wise at 50-55° C. Maintained the reaction mass for 2 hrs at the same temperature and allowed to cool to temperature. Stirred for about 2 hrs at room temperature, filtered and washed with chilled isopropyl alcohol to obtain pure Abacavir sulphate. The compound was confirmed by ¹H-NMR, Mass, IR, SOR and XRD. (Yield: 95%, HPLC: >99.8%).

### Example 19: Preparation of {(1S,4R)-4-[2-amino-6-(cyclopropylamino)-9H-purin-9-yl]cyclopent-2-en-1-yl}methanol sulphate

To a solution of acetone and water was added {(1S,4R)-4-[2-amino-6-(cyclopropylamino)-9H-purin-9-yl]cyclopent-2-en-1-yl}methanol (0.3492 mol) and heated to 50-55° C. Activated carbon was added and filtered through hyflo bed. To the filtered MLs con. Sulphuric acid (0.2 mol) was added drop wise at 50-55° C. Maintained the mass for about 2 hrs at the same temperature and allowed to cool to room temperature. Stirred the reaction mass for about 2 hrs at room temperature filtered and washed with chilled acetone to obtain pure Abacavir sulphate. The compound was confirmed by ¹H-NMR, Mass, IR, SOR and XRD. (Yield: 94%, HPLC: >99.8%).

### Example 20: Preparation of {(1S,4R)-4-[2-amino-6-(cyclopropylamino)-9H-purin-9-yl]cyclopent-2-en-1-yl}methanol sulphate

To a solution of isopropyl alcohol and water was added {(1S,4R)-4-[2-amino-6-(cyclopropylamino)-9H-purin-9-yl]cyclopent-2-en-1-yl}methanol (0.0874 mol). Heated to 55-60° C. Carbon was added (two times) and filtered through hyflo bed and washed the bed with hot isopropyl alcohol. To the filtered MLs sulphuric acid (0.0439 mop in isopropyl alcohol was added drop wise at 45-50° C. Maintained the mass for 7-8 hrs at the same temperature and allowed to room temperature. Stirred for 7-8 hrs at room temperature, further maintained for 60 min at 0-5° C. Filtered the mass and washed with chilled isopropyl alcohol to obtain pure Abacavir sulphate. (yield: 89.5%, HPLC purity: 99.75%).

### Example 21: Preparation of {(1S,4R)-4-[2-amino-6-(cyclopropylamino)-9H-purin-9-yl]cyclopent-2-en-1-yl}methanol sulphate

To a solution of acetone and water was added {(1S,4R)-4-[2-amino-6-(cyclopropylamino)-9H-purin-9-yl]cyclopent-2-en-1-yl}methanol (0.0174 mol). Heated to 50-55° C. Carbon was added and filtered through hyflo bed and washed the bed with hot acetone. To the filtered MLs sulphuric acid (0.0087 mol) in acetone was added drop wise at 25-35° C. Stirred for 30 min at the same temperature, further maintained for 60 min at 0-5° C. Filtered the mass and washed with chilled acetone to obtain pure Abacavir sulphate. (yield: 90%, HPLC purity: 99.16%).

### Example 22: Preparation of {(1S,4R)-4-[2-amino-6-(cyclopropylamino)-9H-purin-9-yl]cyclopent-2-en-1-yl}methanol sulphate

To a solution of ethanol and water was added {(1S,4R)-4-[2-amino-6-(cyclopropylamino)-9H-purin-9-yl]cyclopent-2-en-1-yl}methanol (0.0874 mol). Heated to 50-55° C. Sulphuric acid (0.044 mol) in ethanol was added drop wise at 45-50° C. Maintained the mass for 7-8 hrs at the same temperature and allowed to room temperature. Stirred for 7-8 hrs at room temperature, further maintained for 60 min at 0-5° C. Filtered the mass and washed with chilled ethanol to obtain pure Abacavir sulphate. (yield: 73.5%, HPLC purity: 99.78%).

1. (canceled)

2. An improved process for the preparation of (IS,4R)-4-amino-2- cyclopentene-1 -methanol salt of formula IIIa 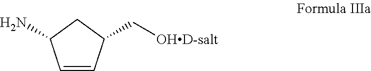
which comprises the steps of
i) converting of compound of formula IVa
to compound of Formula IIIc 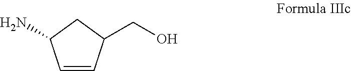
using a suitable reducing agent in the presence of a base and in a solvent,
ii) resolution of compound obtained in step i) using a chiral acid with D-configuration in a solvent and
iii) isolating compound of Formula IIIa.

3. (canceled)

4. (canceled)

5. (canceled)

6. An improved process for the preparation of of (1S,4R)-4-amino-2-cyclopentene-1-methanol D-hydrogen tartrate of Formula IIIb according to claim 21, which comprises the steps of
i) converting compound of formula IVb 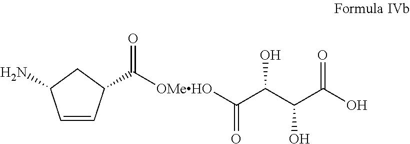
in the presence of a base, and using a suitable reducing agent;
ii) resolution of compound obtained in step i) using a chiral acid with D-configuration in a solvent and
iii) isolating compound of Formula IIIb.

7. An improved process for the preparation of (1S,4R)-4-amino-2-cyclopentene-1-methanol D-hydrogen tartrate of Formula IIIb according to claim 6, which comprises the steps of
i) converting the compound of formula IVb 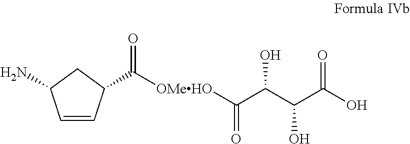
in the presence of sodium hydroxide and using sodium borohydride in n-butanol,
ii) resolution of compound obtained in step i) using D-tartaric acid in methanol and
iii) isolating compound of Formula IIIb.

8. (canceled)

9. An improved process for the preparation of (1S,4R)-4-amino-2-cyclopentene-1-methanol D-hydrogen tartrate of Formula IIIb, which comprises the steps of:
i) reaction of racemic 2-azabicyclo[2.2.1]hept-5-en-3-one 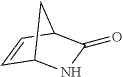
with methanol in the presence of dry HCl gas followed by resolution with L-(+)-tartaric acid in water in the presence of triethylamine give (1S,4R)-methyl-4-amino-2-cyclopentene carboxylate L-hydrogen tartrate of Formula IVb, 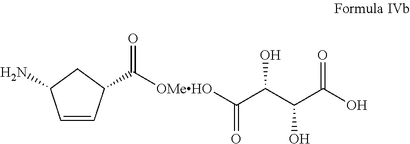
ii) treating compound of formula IVb with solution of sodium hydroxide and sodium borohydride in 2-butanol,
iii) resolution of compound obtained in step iii) using D-tartaric acid in methanol and v) isolating compound of Formula IIIb.

10. (canceled)

11. The process according to claim 2, wherein the base used is selected from alkali metal hydroxides such as sodium hydroxide, lithium hydroxide or potassium hydroxide, or alkali metal carbonates such as sodium carbonate or potassium carbonate, or alkoxides such as sodium methoxide, potassium methoxide, sodium ethoxide, potassium ethoxide.

12. (canceled)

13. The process according to claim 2, wherein reducing agents used is selected from metal hydrides such as boron reagents like sodium borohydride, lithium borohydride, zinc borohydride, sodium cyanoborohydride, sodium triacetoxyborohydride, and the like or aluminum reagents like diisobutylaluminum hydride, aluminum hydride, lithium aluminum hydride.

14. (canceled)

15. The process according to claim 2, wherein solvents are selected from water, methanol, ethanol, propanol, butanol and the solvents used in resolution are selected from methanol, ethanol, isopropanol, butanol, 1,2-dimethoxy ethanol, 2-methoxy ethanol, 2-ethoxy ethanol and ethylene glycol or mixtures thereof.

16. The process according to claim 1, wherein the Chiral acid used for resolution are selected from acids having D-configuration such as malic acid, mandelic acid, tartaric acid, diacetyl tartaric acid, di-p-anisolyl tartaric acid, dibenzoyl tartaric acid, ditoluoyl tartaric acid, camphorsulfonic acid.

17. (canceled)

18. Abacavir (II) which comprises the steps of:
i) reaction of compound of Formula V 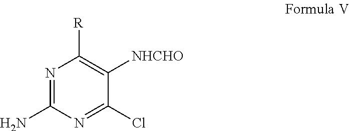
wherein R is halogen selected from chloro, fluoro, bromo, iodo; with compound of Formula IIIa 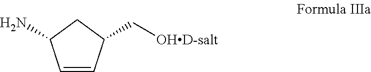
in a solvent in the presence of a base to give compound of Formula VI, 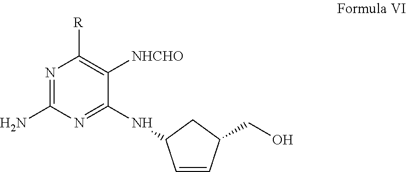
wherein R is halogen selected from chloro, fluoro, bromo, iodo;
ii) cyclization of compound of Formula VI in the presence of triethyl orthoformate to give compound of Formula VII, 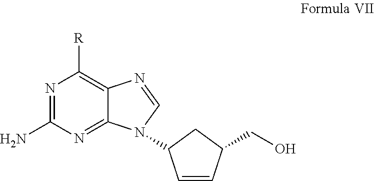
wherein R is halogen selected from chloro, fluoro, bromo, iodo;
iii) conversion of compound of Formula VII to compound of II where in R is cyclopropylamine respectively.

19. The process according to claim 18, wherein the base used in step i) is selected from organic bases such as triethylamine inorganic bases such as alkali metal hydroxides, such as lithium hydroxide, sodium hydroxide, potassium hydroxide; alkaline metal hydroxides such as aluminum hydroxide, magnesium hydroxide, calcium hydroxide; alkali metal carbonates, such as sodium carbonate, potassium carbonate, lithium carbonate, alkali metal bicarbonates, such as lithium bicarbonate, sodium bicarbonate, potassium bicarbonate.

20. The process according to claim 18, wherein the solvent used in step i) is selected from water; alcohols, such as for example, methanol, ethanol, propanol, butanol, pentanol, isopropyl alcohol, 2-butanol, ethylene glycol, glycerol; esters, such as for example, ethyl acetate, propyl acetate, butyl acetate, methyl propanoate, ethyl propanoate, aromatic hydrocarbons, such as for example, toluene, xylene, chlorobenzene, tetralin; nitriles, such as for example, acetonitrile, propionitrile; or any mixtures thereof.

21. An improved process for the preparation of (1S,4R)-4-amino-2-cyclopentene-1-methanol salt according to claim 2, wherein D-Salt is D-tartarate salt and L-salt is L-tartarate salt having the structural formula: [chemical structure]
